(19) United States
(12) Patent Application Publication
(10) Pub. No.: US 2024/0216706 A1
(43) Pub. Date: Jul. 4, 2024

(54) WEARABLE GARMENT WITH INTEGRATED TUMOR TREATING FIELDS VOLTAGE GENERATOR

(71) Applicant: Novocure GmbH, Root D4 (CH)

(72) Inventors: Francis X. LEONARD, Wayne, PA (US); Uri WEINBERG, Haifa (IL)

(73) Assignee: Novocure GmbH, Root D4 (CH)

(21) Appl. No.: 18/393,290

(22) Filed: Dec. 21, 2023

Related U.S. Application Data
(60) Provisional application No. 63/435,636, filed on Dec. 28, 2022.

(51) Int. Cl. A61N 1/40; A41D 1/00; A41D 1/04; H02J 7/00

(52) U.S. Cl. CPC A61N 1/40; A41D 1/002; A41D 1/04; H02J 7/0048; H02J 7/0063; A41D 2200/00; A41D 2400/32

(57) ABSTRACT

A garment configured to generate an AC voltage, the garment including: a support layer configured to be worn on a subject's body; an amplifier for converting an input voltage to the AC voltage; control circuitry communicatively coupled to the amplifier and configured to control the frequency and amplitude of the AC voltage output from the amplifier; and at least one battery coupled to the amplifier and configured to supply the input voltage to the amplifier; wherein the amplifier, the control circuitry, and the at least one battery are integrated into the garment such that the weight of the amplifier, the control circuitry, and the at least one battery is supported by the support layer.

### CROSS-REFERENCE TO RELATED APPLICATIONS

This application claims priority to U.S. Provisional Application No. 63/435,636, filed Dec. 28, 2022, the contents of which are incorporated by reference herein in their entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into the region of interest by transducers placed on the subject's body and applying AC voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment. Conventionally, the AC voltage is applied to the transducers via an AC voltage generator located away from the subject's body and coupled to the transducers via leads.

### BRIEF DESCRIPTION OF THE DRAWINGS

FIG. 1 depicts an example system including a garment with integrated voltage generation components.

FIG. 2 depicts an example vest with integrated voltage generation components.

FIG. 3 depicts an example belt with integrated voltage generation components.

FIG. 4 depicts an example system including a vest and belt with integrated voltage generation components.

FIGS. 5A and 5B are perspective and exploded views depicting an example subassembly having voltage generation components.

FIG. 6 depicts a cross section of a portion of a garment with one or more integrated voltage generation components.

FIG. 7 depicts another example system including a garment with integrated voltage generation components.

### DESCRIPTION OF EMBODIMENTS

This application describes exemplary wearable garments having voltage generation components integrated therein. These wearable garments allow a subject to wear an AC voltage generator on their body.

In general, one or more pairs of transducers are positioned on the subject's body and used to alternately apply TTFields to the subject's body (e.g., on the subject's head, torso, or other locations). The pairs of transducers are electrically coupled via leads to an AC voltage generator, which is conventionally located in an external housing away from the subject's body. For example, the AC voltage generator may be located several feet away from the locations of the transducers positioned on the subject's body. The AC voltage generator is bulky and must be kept near the subject at all times during TTFields treatment. In general, subjects carry the generator around in a backpack or shoulder bag when they are moving about, with the transducers (on the subject's body) tethered to the generator via the leads, and they may tend to leave the AC voltage generator on the floor while they are in one place for a period of time. This continuous attachment to a bulky, external AC voltage generator can be cumbersome and discouraging to subjects, potentially leading subjects to reduce the amount of time they spend using the system for TTFields treatment.

As discovered by the inventors, an AC voltage generator can be integrated into one or more wearable garments, such as a vest, belt, or fanny pack that can be worn against the subject's body for long periods of time. Such wearable garments may be articles of clothing. The wearable garment(s) include voltage generation components such as an amplifier, control circuitry, and one or more batteries, and all of the voltage generation components may be located on or inside the garment(s) and connected together to provide a low-profile AC voltage generator. The exemplary disclosed wearable garment(s) having an integrated AC voltage generator address the drawbacks associated with conventional AC voltage generators. In particular, exemplary embodiments provide a less intrusive AC voltage generator that the subject can more easily incorporate into their daily activities. Using exemplary wearable garment(s) with integrated voltage generation components, subjects will no longer be tethered to a bulky external device that limits or deters free movement, and/or subjects will no longer have to carry a backpack everywhere they go. In addition, no long wires will be hanging from the subject's body since the entire AC voltage generator may be located against the subject's body while they wear the garment(s). The low profile and ease of movement with the inventive wearable garment(s) with integrated voltage generation components may encourage subjects to use the TTFields treatment apparatus for longer time periods, and longer treatment times may be associated with improved treatment outcomes.

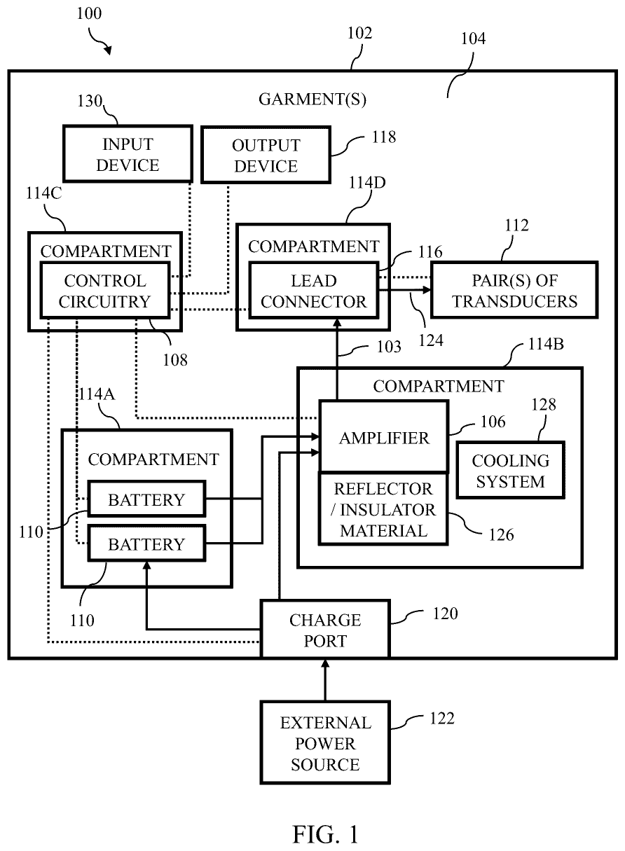

FIG. 1 depicts a system 100 including a garment 102 with voltage generation components integrated therein. The garment 102 may be configured to generate an AC voltage 103 using the voltage generation components. The garment 102 may include a support layer 104 configured to be worn on the subject's body. The support layer 104 may be formed from one or more layers of fabric. The support layer 104 may be configured to support the weight of the voltage generation components incorporated into the garment 102. These voltage generation components may comprise an amplifier 106, control circuitry 108, and one or more batteries 110.

The garment 102 may include the amplifier 106 for converting an input voltage to the AC voltage 103. The AC voltage 103 may be used to generate alternating electric fields between at least one pair of transducers 112. The alternating electric fields may function as TTFields for treating the body of the subject wearing the garment 102. Optionally, at least one pair of transducers 112 may be supported by the garment 102 (FIG. 1), as described further herein.

The garment 102 may include the control circuitry 108 (e.g., on, or comprising, one or more control boards), which may be communicatively coupled to the amplifier 106 and configured to control the frequency and amplitude of the AC voltage 103 output from the amplifier 106. The control circuitry 108 may include one or more printed circuit boards (PCBs), in particular one or more flexible PCBs.

The garment 102 may include at least one battery 110 coupled to the amplifier 106 and configured to supply the input voltage to the amplifier 106. Although the illustrated embodiment includes two batteries 110, it should be noted that other embodiments of the garment 102 may include any other number of batteries 110 (e.g., one, three, four, five, six, seven, eight, or more batteries 110) incorporated therein. Moreover, the batteries 110 need not be the same size (allowing flexibility in terms of duration of use) or be positioned in a single location of the garment 102, but may be located so as to distribute the weight more uniformly on the body. The amplifier 106, the control circuitry 108, and the at least one battery 110 may be integrated into the garment 102 such that the weight of the amplifier 106, the control circuitry 108, and the at least one battery 110 is supported by the support layer 104.

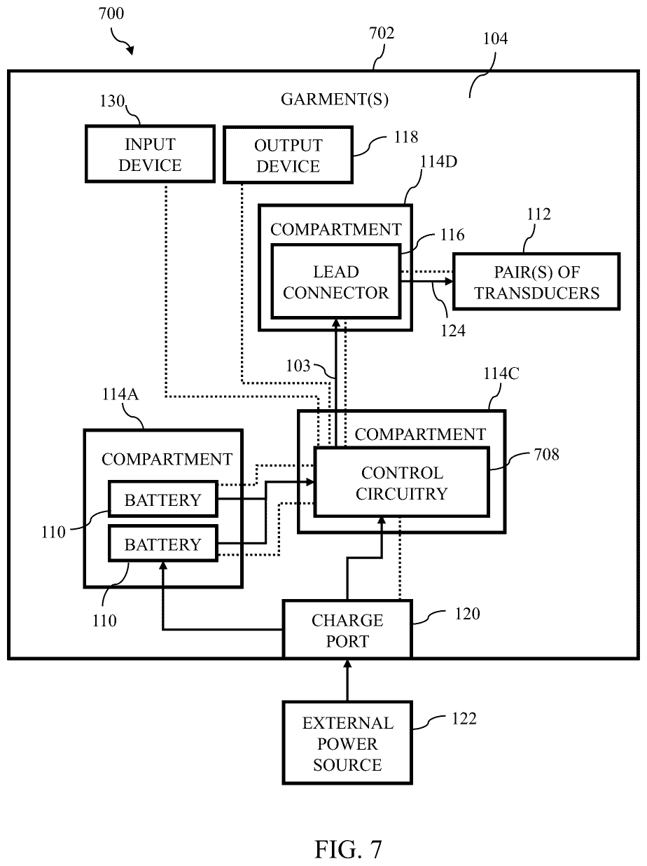

In other embodiments, the amplifier 106 may not be present in the garment 102, and the functions of the amplifier 106 may be performed by the control circuitry 108 (e.g., the control circuitry 708 in FIG. 7). In such embodiments, the control circuitry 108 may be communicatively coupled to the one or more batteries 110 and configured to generate an output voltage (e.g., AC voltage 103). The control circuitry 108 may also be configured to control the frequency and amplitude of the AC voltage 103 output from the control circuitry 108.

Although described as generating an AC voltage 103, it should be noted that in other embodiments the garment 102 may be configured to generate a DC voltage using the integrated voltage generation components.

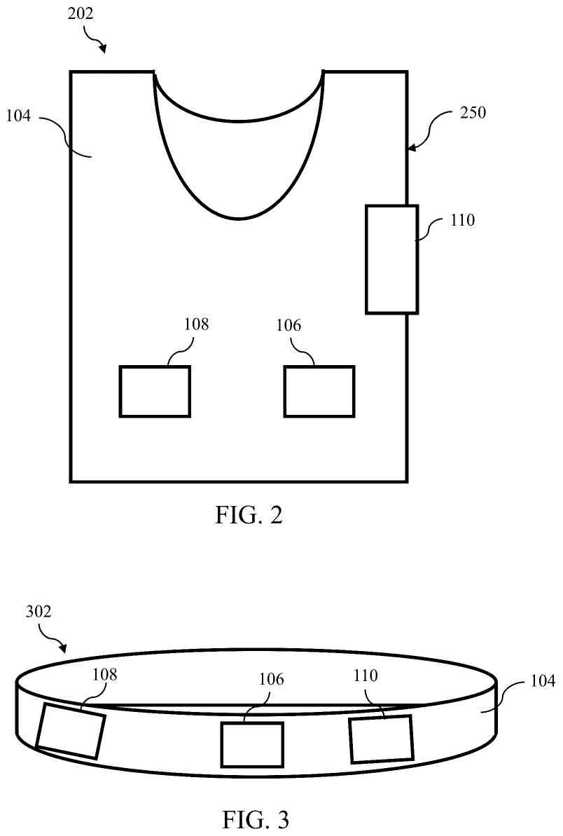

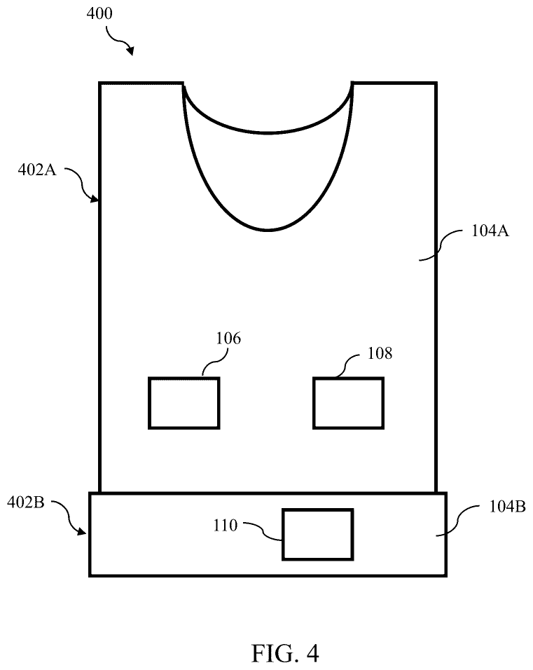

The garment 102 disclosed herein may take many forms. For example, the garment 102 may include a vest, as shown in FIG. 2. In another embodiment, the garment 102 may include a belt or fanny pack, as shown in FIG. 3. In other embodiments, the garment 102 may be modular and/or adapted to be worn on one or more anatomical regions of the subject, which may allow various components of the garment 102 to be attached to one or more anatomical regions of the subject, and such arrangements may result in greater comfort and/or convenience for the subject. In other embodiments, the system 100 may include voltage generation components distributed between multiple garments 102 (e.g., a vest combined with either a belt or fanny pack), such as shown in FIG. 4. In any case, the garment(s) 102 function as a wearable device that can be worn on the subject's body. The garment(s) 102 may be form fitting so as to be worn under the subject's clothes. In other embodiments, the garment(s) 102 may be worn over the subject's clothes, particularly if the garment(s) 102 incorporate a fashionable design on an outward-facing portion of the support layer 104.

The support layer 104 of the garment 102 may include at least one compartment 114 (e.g., 114A, 114B, 114C, and 114D) for holding voltage generation components incorporated into the garment 102. In particular, the at least one compartment 114 (e.g., 114A, 114B, 114C, and 114D) may hold the at least one battery 110, the amplifier 106, and the control circuitry 108 therein. As shown in FIG. 1, for example, the batteries 110 may be located in a first compartment 114A, the amplifier 106 may be located in a second compartment 114B, and the control circuitry 108 may be located in a third compartment 114C. In other embodiments, a single compartment 114 in the support layer 104 may hold a combination of the batteries 110, the amplifier 106, and/or the control circuitry 108 therein. In still other embodiments, one or more of the batteries 110, the amplifier 106, and the control circuitry 108 may be connected to and supported by the support layer 104 without using compartment(s) 114. The control circuitry 108 may include multiple PCBs that are communicatively coupled to each other and distributed throughout different compartments 114 of the garment 102.

As shown in FIG. 1, other components of the AC voltage generator (e.g., a lead connector 116) may be held in one or more compartments (e.g., compartment 114D). In addition, other components of the AC voltage generator (e.g., one or more output devices 118, charge port 120, one or more input devices 130, etc.) may or may not be located in compartments of the support layer 104. The one or more compartments 114 may include rigid or semi-rigid housings, or flexible material compartments sized for the particular component(s) designed to be placed therein. The compartment(s) 114 may be pockets that can be easily accessed by a subject while the subject is wearing the garment 102. In other embodiments, the compartment(s) 114 may be internally secured inside an outer shell of the support layer 104. To avoid having any components moving within a compartment 114, the compartment 114 may have any components within the compartment 114 snuggly situated to avoid movement. As an example, the compartment 114 may include an elastic portion and/or a hook-and-loop portion to secure any components next to the support layer 114.

The one or more batteries 110 of the garment 102 may be low profile batteries. The term “low profile batteries” may refer to batteries taking up less three-dimensional space or being arranged in a flexible manner that allows for easier movement of the subject, compared to the large, cylindrical battery used in conventional AC voltage generators. For example, the batteries 110 may be square shaped and/or thin to be worn less intrusively across the subject's body. As another example, a battery 110 may have a curvature that matches a portion of the subject's body along which the battery 110 would be located when the subject is wearing the garment 102. In another example, one or more batteries 110 may be located in a semi-rigid case, allowing increased flexibility within the garment 102. In another example, multiple smaller batteries 110 may be connected together by a flexible substrate and/or wiring, allowing the battery pack to bend around the subject's body.

The batteries 110 of the garment 102 may be electrically coupled together in series or parallel. The batteries 110 may be stacked in a manner that maintains a relatively low profile of the connected batteries 110. In other embodiments, individual batteries 110 of the garment 102 may be electrically separate from each other, and the control circuitry 108 may control switching between one battery (or group of batteries) 110 and another battery (or group of batteries) 110 for outputting the input voltage to the amplifier 106. As such, one battery 110 (or group of batteries 110) may act as the primary power source for the amplifier 106, and another battery 110 (or group of batteries 110) may act as the back-up battery for the garment 102.

The batteries 110 may be replaceable with new and/or different sized batteries. In particular, the at least one battery 110 may be removably disposed in the support layer 104 and replaceable with at least one other battery 110. The other voltage generation components (e.g., amplifier 106 and control circuitry 108) in the garment 102 may be adaptable to operate with batteries 110 of different voltages installed in the garment 102. As such, the battery size and/or capacity may be easily adjusted to suit the needs of the subject wearing the garment 102.

The control circuitry 108 may be coupled to the at least one battery 110 and configured to monitor a remaining charge of the at least one battery 110. The control circuitry 108 may send a signal to automatically switch from one battery 110 (e.g., a primary battery) to another battery 110 (e.g., back-up battery) to provide the input voltage to the amplifier 106, upon detecting a low charge in the primary battery. Additionally, or alternatively, the control circuitry 108 may send a signal to output an indication of low battery charge to a user. As an example, the garment 102 may include one or more output devices 118. For example, the garment 102 may include an output device 118 communicatively coupled to the control circuitry 108, and the control circuitry 108 may cause the output device 118 to output a low battery indication upon detecting the remaining charge of at least one battery 110 is below a threshold. The output device 118 may be integrated into the garment 102 such that the weight of the output device 118 is supported by the support layer 104. In other embodiments, the output device 118 may be located external to the garment 102 (e.g., integrated into an external power source 122, a user device (e.g., cellular phone), etc.). In other embodiments, the output device 118 may communicate with an off-garment device.

In an example, the output device 118 may include one or more lights and/or a display and the low battery indication may include, for example, a blinking or red light and/or a visual output on the display. In another example, the output device 118 may include a speaker and the low battery indication may include an audible tone, alarm, or message output from the speaker. In another example, the output device 118 may include a haptic-capable device, and the low battery indication may include haptic feedback output to the subject. In another example, the output device 118 may include a Bluetooth device (or other wireless communication device), and the low battery indication may include a message, alarm, or notification sent to a separate user device (e.g., cellular phone) via Bluetooth connection (or other wireless communication).

In an example, the output device 118 may provide output information and/or transmit signals regarding output information. Such output information may include, for example, status of the garment, data related to the performance of the garment, data related to a therapy (e.g., TTFields) being administered to a subject using the garment, data related to therapy or therapeutic parameters associated with a therapy being administered to a subject using the garment, or other information regarding the garment and/or a therapy being administered using the garment. In another example, if the output device 118 wirelessly transmits a signal (e.g., via Bluetooth, Wi-Fi, cellular, personal area network, etc.), the transmitted signal may be received by an off-garment device (e.g., a designated wireless device customized to receive the transmitted signal or a personal mobile device having a mobile app stored thereon). Having the information transmitted from the garment 102 to an off-garment device may increase the wearability and/or convenience of the garment 102. For example, the transmitted signal may be received by a device of the subject, a device of the subject's caregiver, or a device at a distant location monitoring the status of the garment 102. Further, using such an off-garment device may also decrease the weight of the garment 102 by reducing the number of components carried by the garment 102. These examples of various types of output devices 118, off-garment devices, low battery indications, and output information may be used in any combination.

In some embodiments, the control circuitry 108 may receive a signal to start, adjust, or stop generating an alternating electric field (e.g., TTFields). The signal may be initiated by a user (e.g., a subject wearing the garment 102 or a caregiver for the subject wearing the garment 102). As an example, the garment 102 may include one or more input devices 130. For example, the garment may include an input device 130 communicatively coupled to the control circuitry 108, and the control circuitry 108 may receive a signal from the input device 130 to start, adjust, or stop generating an alternating electric field. The input device 130 may be integrated into the garment 102 such that the weight of the input device 130 is supported by the support layer 104. In other embodiments, the input device 130 may be located external to the garment 102 (e.g., integrated into an external power source 122, a user device (e.g., cellular phone), etc.).

In an example, the input device 130 may include one or more buttons or switches and/or a touch-screen display. In an example, the input device 130 may include a Bluetooth device (or other wireless communication device). In an example, the input device 130 and the output device 118 may be integrated into a single device (e.g., a touch-screen display or a Bluetooth device (or other wireless communication device)). These examples of various types of input devices 130 may be used in any combination.

The garment 102 may include a pair of transducers 112 electrically coupled to the amplifier 106. In some embodiments, two pairs of transducers 112 may be located on or electrically coupled to components of the garment 102. Each transducer 112 may include one or more electrode elements. As an example, each transducer 112 may include an array of electrode elements. The pair(s) of transducers 112 may be configured to induce TTFields via the AC voltage 103 output from the amplifier 106 (or, in the embodiment of FIG. 7, via the control circuitry 708). The pair(s) of transducers 112 may be configured for placement on the head of the subject's body, on the torso of the subject's body, or elsewhere on the subject's body. As shown in FIG. 1, the pair(s) of transducers 112 may be integrated into the garment 102 such that the weight of the pair(s) of transducers 112 is supported by the support layer 104. To that end, the pair(s) of transducers 112 may be integrated into the garment 102 at particular locations that enable the pair(s) of transducers 112 to apply alternating electric fields (e.g., TTFields) at a desired frequency and intensity to a target region (e.g., tumor) within the subject's body (e.g., within the torso of the subject's body). Examples of pairs of transducers being integrated into a garment are provided in U.S. patent application Ser. No. 18/062,372, filed Dec. 6, 2022, U.S. patent application Ser. No. 18/062,421, filed Dec. 6, 2022, and U.S. patent application Ser. No. 18/063,464, filed Dec. 8, 2022, each of which are incorporated by reference herein. In other embodiments, one or more pairs of transducers 112 may be located external to the garment 102 (e.g., free-standing and/or attached directly to the subject's body) but electrically coupled to the garment 102.

The garment 102 may include a lead connector 116 electrically coupled to the amplifier 106 and configured to transmit the AC voltage 103 to leads 124 for the pair of transducers 112. For example, in some embodiments, the voltage generation components supply the transducers with an electrical signal having an alternating current waveform at frequencies in a range from about 50 kHz to about 1 MHz. The lead connector 116 may be integrated into the garment 102 such that the weight of the lead connector 116 is supported by the support layer 104. The lead connector 116 may include control and/or monitoring circuitry. The lead connector 116 may be in communication with one or more sensors (e.g., temperature sensors) on the pair of transducers 112. In other embodiments, the lead connector 116 may simply function as an electrical connection point without providing additional circuitry, and the control circuitry 108 may execute all control and/or monitoring operations. In embodiments where the pair of transducers 112 are external to the garment 102, the lead connector 116 may be located at a surface of the support layer 104 (e.g., attached to support layer 104 or located in a compartment 114, such as, for example, 114D of the support layer 104) to enable simple connection of the leads 124 from the transducers into the AC voltage generator in the garment 102. In some embodiments, the garment 102 may not include a lead connector 116 and instead may have the leads 124 connected directly to the amplifier 106.

The garment 102 may include the leads 124 for coupling the amplifier 106 to the pair of transducers 112, and the leads 124 may be located within the support layer 104 such that the leads 124 do not contact the subject's skin while the subject is wearing the garment 102. Other types of wiring (e.g., between the batteries 110 and the amplifier 106, between the charge port 120 and the batteries 110, between the charge port 120 and the amplifier 106, and/or between the amplifier 106 and the lead connector 116) of the garment 102 may be located within the support layer 104 such that the wiring does not contact the subject's skin while the subject is wearing the garment 102.

As illustrated, the system 100 may include an external power source 122 that is separate from and connectable to the garment 102. This external power source 122 may include a larger external battery pack that the voltage generation components of the garment 102 may plug into as a charger or power source. In another example, the external power source 122 may be any standard grid connection (e.g., a wall outlet).

The garment 102 may include the charge port 120 coupled to the amplifier 106. The charge port 120 may be integrated into the garment 102 such that the weight of the charge port 120 is supported by the support layer 104. The charge port 120 may be selectively coupled to the external power source 122. The charge port 120 may be the only component of the AC voltage generator that extends outside the support layer 104 of the garment 102 (e.g., to connect to the external power source 122). As shown in FIG. 1, the charge port 120 may be connected to the at least one battery 110 as well. Having the charge port 120 connected to the at least one battery 110 may allow the external power source 122 to power the transducers 112 directly by providing the input voltage to the amplifier 106 (battery override) and/or to charge the batteries 110 in the garment 102 (battery charging). The charge port 120 may further be coupled to the control circuitry 108. The control circuitry 108 may be configured to selectively direct power from the charge port 120 to the at least one battery 110 for charging the at least one battery 110 and/or to the amplifier 106 for supplying the input voltage.

The charge port 120 may include a breakaway connector that allows the charge port 120 to be disconnected from the external power source 122 with a small amount of force (e.g., in response to the subject moving away from the external power source 122). The charge port 120 may include a magnetic connection, for example, to the external power source 122 that enables the charge port 120 to remain securely plugged into the external power source 122 until the subject moves away from the external power source 122, triggering the breakaway function.

The amplifier 106 may generate the most heat of all the voltage generation components in the garment 102. As such, the garment 102 may be equipped with features to protect the subject from excess heat and/or to keep the amplifier 106 and other components in the garment 102 from overheating. For example, the garment 102 may include a reflective material or an insulating material 126 located between the amplifier 106 and a skin-facing portion of the support layer 104 to protect the subject's skin from the heat generated by the amplifier 106. In the embodiment of FIG. 7, one or more portions of the control circuitry 708 may generate the most heat, and the garment 102 may include a similar reflective material or insulating material located between these one or more portions of the control circuitry 708 and a skin-facing portion of the support layer 104 to protect the subject's skin from excess heat. A similar reflective material or insulating material may be located between any system components that generate an uncomfortable amount of heat and a skin-facing portion of the support layer 104.

Turning back to FIG. 1, the garment 102 may include a cooling system 128 configured to cool the amplifier 106. The cooling system 128 may be integrated into the garment 102 such that the weight of the cooling system 128 is supported by the support layer 104. The cooling system 128 may include, for example, a passive cooling system such as a heat sink, or an active cooling system such as a fan and/or coolant circulating system. An active cooling system may be coupled to the control circuitry 108, where the control circuitry 108 may provide control signals to the active cooling system, and/or the battery 110, where the battery 110 may provide power to the active cooling system. In the embodiment of FIG. 7, one or more portions of the control circuitry 708 may generate the most heat, and the garment 102 may include a similar cooling system configured to cool these one or more portions of the control circuitry 708. The same or a similar cooling system may be used to cool any system components that generate an amount of heat requiring dissipation (e.g., a large amount of heat, or an uncomfortable amount of heat).

In some embodiments, the garment 102 may be equipped with relatively small batteries 110 that carry a limited charge (e.g., only 15-30 minutes of charge). Such a small size may decrease the weight of the batteries 110 on the support layer 104, and ultimately on the subject wearing the garment 102. In such instances, the batteries 110 may be rechargeable by plugging the garment 102 into the external power source 122 via the charge port 120 on the garment 102, as described above. Having small batteries 110 may allow a subject to unplug from the external power source 122 for a certain length of time to do something without remaining in constant contact to a large battery bank or a wall outlet. In an example, a sufficient number of small batteries may be carried on the garment 102 to sustain power to the garment during one or more activities of the subject. As such, having small batteries 110 may provide increased mobility and ease of use for the subject wearing the garment 102.

As discussed above, the size, number, and capacity of batteries 110 used in the garment 102 may be adjusted. In certain embodiments, the support layer 104 may include multiple compartments 114 of different sizes, each one configured to hold a different number of batteries 110 or different sized batteries 110. Having such multiple compartments 114 of different sizes may allow for simple switching out of batteries when a larger or smaller battery capacity is desired. The number and weight of batteries 110 that may be supported in the garment 102 may allow for individual subjects to customize their garment 102 based on the amount of weight they wish to carry around and desired battery life.

Like the batteries 110, the amplifier 106 and control circuitry 108 may be removable from the support layer 104 (e.g., via removal from compartments 114) to allow for easy maintenance, repair, or replacement of faulty equipment, as well as to enable the support layer 104 to be washed.

FIG. 2 depicts a garment 202 taking the form of a vest. The term “vest” may include any article of clothing that can be worn on the torso. The vest may or may not have sleeves at the arm holes. The vest may include an opening in the middle of its front (with clasps, buttons, a zipper, hook and loop fasteners, etc.) to make it easier for the subject to put on and fasten the garment 202. The vest may be a compression garment. As illustrated, the garment 202 may include the support layer 104, the amplifier 106, the control circuitry 108, and at least one battery 110, as discussed at length above. The garment 202 may also include any other features of the garment 102 discussed above with reference to FIG. 1. As shown in FIG. 2, the various components of the integrated AC generator may be distributed throughout different locations of the vest, for example, to balance the weight of the garment 202 across the subject's body and make the garment 202 lower profile. As illustrated, the at least one battery 110 may be disposed at a location of the support layer 104 under an arm hole 250 of the vest (202). The battery 110 may be the largest component of the AC voltage generator, and therefore the position under the arm hole 250 may provide increased space for the battery 110 while keeping a low profile of the vest.

FIG. 3 depicts a garment 302 taking the form of a belt (or fanny pack). In some embodiments, the belt may take the form of a tool belt with multiple compartments located around the belt. As illustrated, the garment 302 may include the support layer 104, the amplifier 106, the control circuitry 108, and at least one battery 110, as discussed at length above. The garment 302 may also include any other features of the garment 102 discussed above with reference to FIG. 1. As shown in FIG. 3, the various components of the integrated AC generator may be distributed throughout different locations of the belt, for example, to balance the weight of the garment 302 across the subject's body and make the garment 302 lower profile. In some embodiments, the garment 302 may take the form of a fanny pack having the largest component(s) (e.g., battery 110) in the main compartment. In other embodiments, the garment 302 may take the form of a fanny pack having the amplifier 106, which runs hottest and needs to be kept cooler, in the main compartment so as to keep the amplifier 106 further away from the subject's skin. The belt/fanny pack style garment 302 may be worn around the waist or across the torso (e.g., over one shoulder) of the subject's body.

FIG. 4 depicts a system 400 including multiple garments 402A and 402B with voltage generation components integrated therein. The system 400 may be configured to generate an AC voltage. The first garment 402A may be or may include a vest configured to be worn on the subject's body, and the second garment 402B may be or may include a belt or fanny pack configured to be worn on the subject's body. The system 400 may include, as discussed above with reference to FIG. 1: the amplifier 106 for converting an input voltage to the AC voltage; control circuitry 108 communicatively coupled to the amplifier 106 and configured to control the frequency and amplitude of the AC voltage output from the amplifier 106; and at least one battery 110 coupled to the amplifier 106 and configured to supply the input voltage to the amplifier 106. The amplifier 106, control circuitry 108, and at least one battery 110 may be integrated into the first and second garments 402A and 402B such that the weight of the amplifier 106, control circuitry 108, and at least one battery 110 is supported by the combination of the vest and the belt or fanny pack. For example, as shown in FIG. 4, the amplifier 106 may be disposed in the vest (402A), at least a portion of the control circuitry 108 may be disposed in the vest (402A), and the at least one battery 110 may be disposed in the belt or fanny pack (402B). In other embodiments, the amplifier 106 may be disposed in the belt or fanny pack (402B), at least a portion of the control circuitry 108 may be disposed in the vest (402A), and the at least one battery 110 may be disposed in the vest (402A). Other embodiments may include other combinations of voltage generation components and locations (e.g., disposed in the vest 402A and the belt or fanny pack 402B).

As illustrated, the garment 402A may include a support layer 104A, the amplifier 106, and the control circuitry 108, and the garment 402B may include a support layer 104B and at least one battery 110. These voltage generation components may operate as discussed above with reference to FIG. 1. One or both of the garments 402A, 402B may also contain any other features of the garment 102 discussed above with reference to FIG. 1. Although shown as being located in the vest garment 402A, the control circuitry 108 may be located in the belt garment 402B in other embodiments, or the control circuitry 108 may be distributed between both garments 402A and 402B (e.g., with at least one PCB located on each of the vest (402A) and the belt or fanny pack (402B)). One or more leads may extend between the component(s) on the first garment 402A and the component(s) on the second garment 402B. These leads may extend from one garment to a connector located on the other garment. In some embodiments, the first garment 402A may include all components of the AC generator (e.g., the amplifier 106, the control circuitry 108, and one or more primary batteries 110), and the second garment 402B may contain one or more back-up batteries 110 that may be switched over to by the control circuitry 108 when the one or more primary batteries 110 are discharged, or nearly discharged. In some embodiments, the first garment 402A may include all components of the AC generator (e.g., the amplifier 106, the control circuitry 108, and the one or more batteries 110 that are currently in use), and the second garment 402B may contain one or more spare batteries that may be traded out for the batteries 110 by the subject once the batteries 110 are discharged. In some embodiments, one or more spare batteries (e.g., in the second garment 402B) may not be electrically connected to the components of the AC generator.

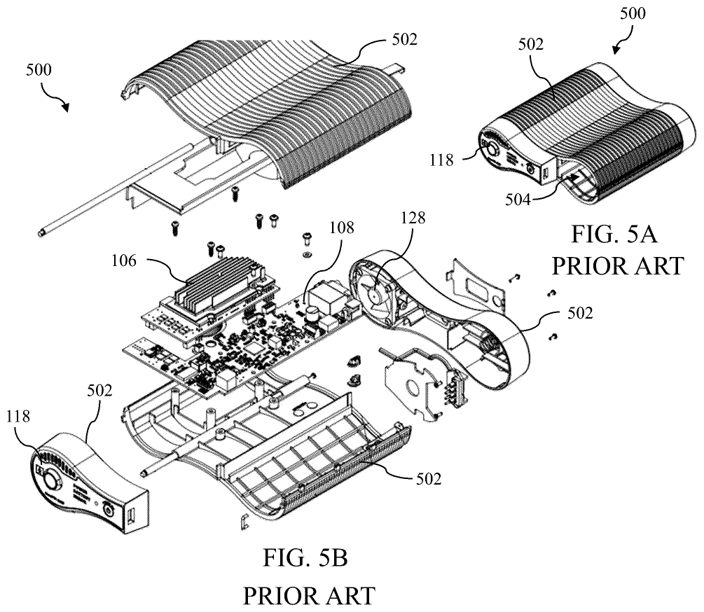

FIGS. 5A and 5B depict an example subassembly 500 having certain components of the AC voltage generator that may be integrated into the garment(s) described above. The illustrated subassembly 500 may include the amplifier 106 and control circuitry 108 discussed above. In addition, the subassembly 500 may include a housing 502 forming a battery compartment 504 configured to receive a battery (e.g., 110 of FIG. 1). The illustrated subassembly 500 also includes an output device 118 and a cooling system 128 (e.g., fan). The subassembly 500 of FIGS. 5A and 5B may be configured to include all three of the amplifier 106, the control circuitry 108, and the battery (110). These voltage generation components may be similarly packaged together within a wearable garment (e.g., 102 of FIGS. 1-3). However, due to the large size, rigidity, and resulting bulkiness of the subassembly 500 of FIGS. 5A and 5B, it may be desirable to break up the voltage generation components into multiple separate locations (e.g., compartments 114) within the garment(s) (102). For example, the voltage generation components may be located in multiple smaller subassemblies located about the garment(s) 102. Having multiple smaller subassemblies may allow for a more even distribution of the weight of the voltage generation components across the subject's body, as well as a lower profile so that the AC voltage generator is not visible or in the way. Distributing the voltage generation components over multiple locations in the garment(s) (102) may also improve flexibility in switching out batteries for different sized batteries (e.g., for adjusting the battery capacity of the AC voltage generator).

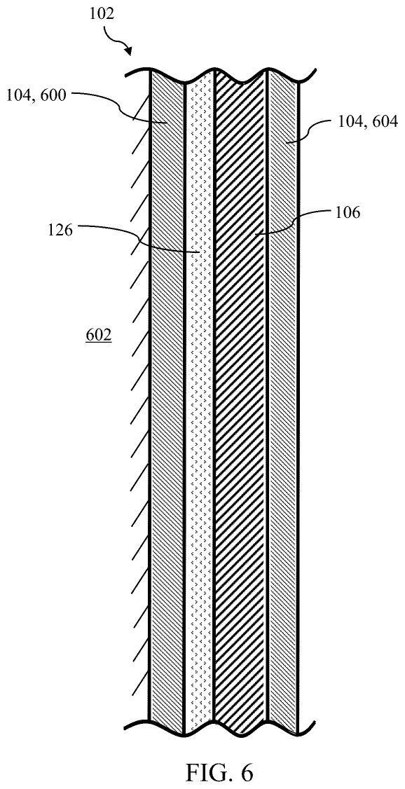

FIG. 6 depicts a cross section of a portion of the garment 102 having the amplifier 106 and a reflective material or an insulating material 126 located between the amplifier 106 and a skin-facing portion 600 of the support layer 104. FIG. 6 illustrates the relative placement of these components (shown as layers) within the garment 102 with respect to a skin layer 602 of the subject's body. It should be understood that the cross section of FIG. 6 is exemplary, and the exact shape, size, and dimensions of these layers of components may differ. Additional layers of components, materials, and/or space may be located in this portion of the garment 102 as well. In embodiments without an amplifier (e.g., as shown in FIG. 7), a portion of the control circuitry 708 that generates heat may be further separated from the subject by using the reflective material or insulating material 126. For example, the reflective material or insulating material 126 may be located between the skin-facing portion 600 and the control circuitry 708 (in place of the amplifier 106 shown in FIG. 6).

The skin-facing portion 600 of the support layer 104 may be continuous with an outward-facing portion 604 of the support layer 104 that faces away from the subject's skin. In other embodiments, the skin-facing portion 600 may form an inner support layer with the outward-facing portion 604 being an outer shell positioned over and attached to the inner support layer. The outward-facing portion 604 of the support layer 104 may be removable and replaceable with another outward-facing portion (e.g., having a different color, design, pattern, material, shape, and/or style) to change the outer appearance of the garment 102.

In some embodiments, at least the skin-facing portion 600 of the support layer 104 may be washable. For example, the skin-facing portion 600 of the support layer 104 may be removable for washing. As another example, the skin-facing portion 600 and outward-facing portion 604 may be removable from internal compartments holding the voltage generator components for washing. As another example, the voltage generator components may be removable from the support layer 104 so that all parts of the support layer (including compartments) may be washed.

FIG. 7 depicts an example system 700 including a garment 702 with voltage generation components integrated therein. The garment 702 may be configured to generate an output voltage (e.g., AC voltage 103) using the voltage generation components. The garment 702 may include a support layer 104 configured to be worn on the subject's body and configured to support the weight of the voltage generation components incorporated into the garment 702. These voltage generation components may include, for example, at least control circuitry 708 (which may provide output signals to one more output devices 118 and/or receive input signals from one or more input devices 130) and one or more batteries 110. These voltage generation components may function similarly (or differently) to those described above with reference to FIG. 1. The control circuitry 708 may convert an input voltage to the output voltage and may also control the frequency and amplitude of an AC voltage 103 output from the control circuitry 708. The control circuitry 708 may additionally provide any amplification needed to generate an output voltage (e.g., AC voltage 103). With the control circuitry 708, the function of an amplifier (e.g., amplifier 106 of FIG. 1) may be incorporated into the control circuitry 708, and a separate amplifier (e.g., amplifier 106 of FIG. 1) may not be needed. The at least one battery 110 may be configured to supply the input voltage to the control circuitry 708. The support layer 104 of the garment 702 may include, for example: voltage generation components (e.g., the control circuitry 708, and at least one battery 110); at least one compartment 114 (e.g., 114A, 114C, and 114D) for holding the voltage generation components incorporated into the garment 702; an output device 118 communicatively coupled to the control circuitry 708; a pair of transducers 112 electrically coupled to the control circuitry 708; a lead connector 116 electrically coupled to the control circuitry 708 and configured to transmit the output voltage to leads 124 for the pair of transducers 112; the leads 124 for coupling the control circuitry 708 to the pair of transducers 112; a charge port 120 coupled to the control circuitry 708 and which may be selectively coupled to an external power source 122; an input device 130 communicatively coupled to the control circuitry 708; or any combination thereof. The previously described voltage generation components and configurations of FIGS. 1-6 may be combined with the embodiment of FIG. 7, except where mutually exclusive.

### Illustrative Embodiments

The invention includes other illustrative embodiments (“Embodiments”) as follows.
Embodiment 1: A garment configured to generate an AC voltage, the garment comprising: a support layer configured to be worn on a subject's body; an amplifier for converting an input voltage to the AC voltage; control circuitry communicatively coupled to the amplifier and configured to control the frequency and amplitude of the AC voltage output from the amplifier; and at least one battery coupled to the amplifier and configured to supply the input voltage to the amplifier; wherein the amplifier, the control circuitry, and the at least one battery are integrated into the garment such that the weight of the amplifier, the control circuitry, and the at least one battery is supported by the support layer.
Embodiment 2: The garment of Embodiment 1, wherein the garment comprises a vest.
Embodiment 3: The garment of Embodiment 2, wherein one or more of the at least one battery is disposed at a location of the support layer under an arm hole of the vest.
Embodiment 4: The garment of Embodiment 1, wherein the garment comprises a belt or fanny pack.
Embodiment 5: The garment of Embodiment 1, wherein the support layer comprises at least one compartment holding one or more of the at least one battery, the amplifier, and/or the control circuitry therein.
Embodiment 6: The garment of Embodiment 1, wherein one or more of the at least one battery is removably disposed in the support layer and is replaceable with at least one other battery.
Embodiment 7: The garment of Embodiment 1, wherein the control circuitry is coupled to the at least one battery and configured to monitor a remaining charge of the at least one battery.
Embodiment 8: The garment of Embodiment 7, further comprising an output device communicatively coupled to the control circuitry, wherein the control circuitry is configured to cause the output device to output a low battery indication upon detecting that the remaining charge of the at least one battery is below a threshold.
Embodiment 9: The garment of Embodiment 1, wherein at least a skin-facing portion of the support layer is washable.
Embodiment 10: The garment of Embodiment 1, further comprising a pair of transducers electrically coupled to the amplifier, the pair of transducers configured to induce tumor treating fields (TTFields) via the AC voltage output from the amplifier, the pair of transducers being integrated into the garment such that the weight of the pair of transducers is supported by the support layer.
Embodiment 11: The garment of Embodiment 1, further comprising a lead connector electrically coupled to the amplifier and configured to transmit the AC voltage to leads for a pair of transducers and to receive sensor signals from the pair of transducers, the lead connector being integrated into the garment such that the weight of the lead connector is supported by the support layer.
Embodiment 12: The garment of Embodiment 1, further comprising leads for coupling the amplifier to a pair of transducers, the leads being located within the support layer such that the leads do not contact the subject's skin while the subject is wearing the garment.
Embodiment 13: The garment of Embodiment 1, further comprising a charge port coupled to the amplifier, the charge port being integrated into the garment such that the weight of the charge port is supported by the support layer.
Embodiment 14: The garment of Embodiment 13, wherein the charge port is coupled to the at least one battery and the control circuitry, wherein the control circuitry is configured to selectively direct power from the charge port to the at least one battery for charging the at least one battery or to the amplifier for supplying the input voltage.
Embodiment 15: The garment of Embodiment 13, wherein the charge port comprises a breakaway connector.
Embodiment 16: The garment of Embodiment 1, further comprising a reflective material or an insulating material located between the amplifier and a skin-facing portion of the support layer.
Embodiment 17: The garment of Embodiment 1, further comprising a cooling system configured to cool the amplifier, the cooling system being integrated into the garment such that the weight of the cooling system is supported by the support layer.
Embodiment 18: A system configured to generate an AC voltage, comprising: a first garment comprising a vest configured to be worn on a subject's body; a second garment comprising a belt or fanny pack configured to be worn on the subject's body; an amplifier for converting an input voltage to the AC voltage; control circuitry communicatively coupled to the amplifier and configured to control the frequency and amplitude of the AC voltage output from the amplifier; and at least one battery coupled to the amplifier and configured to supply the input voltage to the amplifier; wherein the amplifier, the control circuitry, and the at least one battery are integrated into the first and second garments such that the weight of the amplifier, the control circuitry, and the at least one battery is supported by the combination of the vest and the belt or fanny pack.
Embodiment 19: The system of Embodiment 18, wherein the amplifier is disposed in the vest, at least a portion of the control circuitry is disposed in the vest, and one or more of the at least one battery is disposed in the belt or fanny pack.
Embodiment 20: The system of Embodiment 18, wherein the amplifier is disposed in the belt or fanny pack, at least a portion of the control circuitry is disposed in the vest, and one or more of the at least one battery is disposed in the vest.
Embodiment 21: A system comprising: a garment configured to generate an output voltage, the garment comprising: a support layer configured to be worn on a subject's body; at least one battery configured to supply an input voltage to the system; and control circuitry communicatively coupled to the battery and configured to generate the output voltage; wherein the at least one battery and the control circuitry are integrated into the garment such that the weight of the at least one battery and the control circuitry is supported by the support layer.
Embodiment 22: The system of Embodiment 21, wherein the output voltage is an AC voltage.
Embodiment 23: The system of Embodiment 22, wherein the control circuitry is configured to control the frequency and amplitude of the AC voltage output from the control circuitry.
Embodiment 24: The system of Embodiment 22, further comprising a pair of transducers electrically coupled to the control circuitry, the pair of transducers configured to induce tumor treating fields (TTFields) via the AC voltage output from the control circuitry, the pair of transducers being integrated into the garment such that the weight of the pair of transducers is supported by the support layer.
Embodiment 25: The system of Embodiment 22, further comprising a lead connector electrically coupled to the control circuitry and configured to transmit the AC voltage to leads for a pair of transducers and to receive sensor signals from the pair of transducers, the lead connector being integrated into the garment such that the weight of the lead connector is supported by the support layer.
Embodiment 26: The system of Embodiment 21, further comprising a charge port coupled to the control circuitry and to the at least one battery, the charge port being integrated into the garment such that the weight of the charge port is supported by the support layer, wherein the control circuitry is configured to selectively direct power from the charge port to the at least one battery for charging the at least one battery or to the control circuitry for supplying the input voltage.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A garment configured to generate an AC voltage, the garment comprising:
a support layer configured to be worn on a subject's body;
an amplifier for converting an input voltage to the AC voltage;
control circuitry communicatively coupled to the amplifier and configured to control the frequency and amplitude of the AC voltage output from the amplifier; and
at least one battery coupled to the amplifier and configured to supply the input voltage to the amplifier;
wherein the amplifier, the control circuitry, and the at least one battery are integrated into the garment such that the weight of the amplifier, the control circuitry, and the at least one battery is supported by the support layer.

2. The garment of claim 1, wherein the garment comprises a vest.

3. The garment of claim 2, wherein one or more of the at least one battery is disposed at a location of the support layer under an arm hole of the vest.

4. The garment of claim 1, wherein the garment comprises a belt or fanny pack.

5. The garment of claim 1, wherein the support layer comprises at least one compartment holding one or more of the at least one battery, the amplifier, and/or the control circuitry therein.

6. The garment of claim 1, wherein one or more of the at least one battery is removably disposed in the support layer and is replaceable with at least one other battery.

7. The garment of claim 1, wherein the control circuitry is coupled to the at least one battery and configured to monitor a remaining charge of the at least one battery.

8. The garment of claim 7, further comprising an output device communicatively coupled to the control circuitry, wherein the control circuitry is configured to cause the output device to output a low battery indication upon detecting that the remaining charge of the at least one battery is below a threshold.

9. The garment of claim 1, wherein at least a skin-facing portion of the support layer is washable.

10. The garment of claim 1, further comprising a pair of transducers electrically coupled to the amplifier, the pair of transducers configured to induce tumor treating fields (TTFields) via the AC voltage output from the amplifier, the pair of transducers being integrated into the garment such that the weight of the pair of transducers is supported by the support layer.

11. The garment of claim 1, further comprising a lead connector electrically coupled to the amplifier and configured to transmit the AC voltage to leads for a pair of transducers and to receive sensor signals from the pair of transducers, the lead connector being integrated into the garment such that the weight of the lead connector is supported by the support layer.

12. The garment of claim 1, further comprising leads for coupling the amplifier to a pair of transducers, the leads being located within the support layer such that the leads do not contact the subject's skin while the subject is wearing the garment.

13. The garment of claim 1, further comprising a charge port coupled to the amplifier, the charge port being integrated into the garment such that the weight of the charge port is supported by the support layer.

14. The garment of claim 13, wherein the charge port is coupled to the at least one battery and the control circuitry, wherein the control circuitry is configured to selectively direct power from the charge port to the at least one battery for charging the at least one battery or to the amplifier for supplying the input voltage.

15. The garment of claim 13, wherein the charge port comprises a breakaway connector.

16. The garment of claim 1, further comprising a reflective material or an insulating material located between the amplifier and a skin-facing portion of the support layer.

17. The garment of claim 1, further comprising a cooling system configured to cool the amplifier, the cooling system being integrated into the garment such that the weight of the cooling system is supported by the support layer.

18. A system configured to generate an AC voltage, comprising:
a first garment comprising a vest configured to be worn on a subject's body;
a second garment comprising a belt or fanny pack configured to be worn on the subject's body;
an amplifier for converting an input voltage to the AC voltage;
control circuitry communicatively coupled to the amplifier and configured to control the frequency and amplitude of the AC voltage output from the amplifier; and
at least one battery coupled to the amplifier and configured to supply the input voltage to the amplifier;
wherein the amplifier, the control circuitry, and the at least one battery are integrated into the first and second garments such that the weight of the amplifier, the control circuitry, and the at least one battery is supported by the combination of the vest and the belt or fanny pack.

19. The system of claim 18, wherein the amplifier is disposed in the vest, at least a portion of the control circuitry is disposed in the vest, and one or more of the at least one battery is disposed in the belt or fanny pack.

20. The system of claim 18, wherein the amplifier is disposed in the belt or fanny pack, at least a portion of the control circuitry is disposed in the vest, and one or more of the at least one battery is disposed in the vest.
